(19) United States
(12) Patent Application Publication
(10) Pub. No.: US 2026/0306029 A1
(43) Pub. Date: Oct. 8, 2026

(54) SYSTEMS AND METHODS FOR CONTINUOUS ERGONOMIC RISK ESTIMATION AND MITIGATION

(71) Applicant: Movella Holdings B.V., Enschede (NL)

(72) Inventors: Domitille PRINCELLE, Enschede (NL); Uthvag SAKTHIVELU, Enschede (NL)

(21) Appl. No.: 19/173,470

(22) Filed: Apr. 8, 2025

(51) Int. Cl. A61B 5/00; A61B 5/103; A61B 5/11

(52) U.S. Cl. CPC A61B 5/7275; A61B 5/1036; A61B 5/1123; A61B 5/486; A61B 5/6807; A61B 2503/20; A61B 2562/0247; A61B 2562/043

(57) ABSTRACT

A system for injury reduction includes a motion capture system worn by a user having motion sensors, for capturing motion data of portions of the user's body when performing physical actions, a smart insole in the user's shoe or smart gloves for measuring a plurality of foot pressures or hand-related loads when performing the physical actions, and a system coupled to the motion capture system and the smart insole, wherein the system is for determining kinematic data associated with the user's body in response to the motion data and a user biomechanical model, wherein the system is for determining physical loadings for other portions of the user's body in response to the plurality of foot pressures or hand-related loads and the kinematic data, wherein the system is for determining whether the physical loadings exceeds a threshold, and if so, the system is for outputting corrective physical actions to the user.

### BACKGROUND

The present invention relates to motion studies for protecting workers. Motion studies within a factory setting typically involve analyzing the movements of workers to identify inefficiencies and improve productivity. By systematically observing and recording the steps involved in various tasks, motion studies can help streamline processes, reduce waste, and enhance worker efficiency. Additionally, motion studies can be used to protect workers from injuries by identifying repetitive motions and awkward postures that can strain their bodies, joints, and muscles. Based on these studies, ergonomic improvements can promote work safety by reducing the risk of work-related musculoskeletal disorders. The benefits of motion studies thus include improved worker safety and higher quality of output. By recommending how tasks should be performed, factories can achieve gains in efficiency and competitiveness.

Conducting reliable motion studies is challenging. One major issue is the problem of worker observation, where the presence of an observer and a test environment often alters how workers perform tasks, leading to inaccurate data. Another issue is segmenting or breaking down a worker's motions while performing a complex task into smaller, measurable tasks. Different workers may perform complex tasks in various ways, and observers or researchers may have different criteria for segmenting the task. This variability in performance and researcher judgment makes it challenging to study worker performance accurately. Addressing these challenges is desirable for the success of motion studies and the implementation of their findings.

In light of the above, what is desired are methods and apparatus for addressing the problem discussed herein, with reduced drawbacks.

### SUMMARY

The present invention relates to methods and apparatus for continuous ergonomic risk estimation and mitigation. More specifically, embodiments include the use of motion sensors for capture of motion data from different portions of a user's body and the use of pressure sensors for the hands or feet of the user to capture load data of the user. A system processes these kinematic data along with a biomechanical model of the user to determine kinematic data as well as load data upon other portions of the user's body. Based upon such actual and computed kinematic data and load data, one or more ergonomic risk computations may be performed and then compared to known ergonomic risk factors. In some cases, when the ergonomic risk factors are exceeded, the system may notify the user directly, along with guidance on how to reduce the risk of being injured. In other cases, a movement specialist, or the like may be notified, and that movement specialist may direct a change to the work environment or work flow for the user, to reduce the user risk.

In some embodiments, the system may capture a stream of motion and pressure data of a user as they perform a specific action repeatedly, as they perform a number of different actions, and the like. As discussed above, manually segmenting of such motion and pressure data and attributing them to specific user motions is very time consuming and is not user friendly. To facilitate real-time or near real-time processing of the ergonomic risk factors, above, the system is enabled to automatically determine time periods where specific motions are performed. In some embodiments, automatic segmentation may be performed based upon the identification of time periods with dramatically different loads on the user's hands and/or the feet.

According to one aspect, a system for injury reduction is described. One apparatus may include a motion capture system worn by a user comprising a plurality of motion sensors, wherein the motion capture system is configured to capture motion data associated with portions of a body of the user in response to the user performing a plurality of physical actions. A system may include a smart insole coupled to a shoe worn by the user, comprising at least one pressure sensor, wherein the one pressure sensor is configured to measure a plurality of pressures associated with a foot of the user in response to the user performing the plurality of physical actions. A system might also include smart gloves comprising at least one pressure sensor, wherein sensor is configured to measure a plurality of pressures associated with hand or fingers of the user in response to the user performing the plurality of physical actions. A device may include a system coupled to the motion capture system and the smart insole, wherein the system is configured to store a biomechanical model of the user, wherein the system is configured to determine a plurality of kinematic data associated with the portions of the body of the user in response to the motion data associated with portions of the body and the biomechanical model, wherein the system is configured to determine a plurality of physical loadings associated with joints of the body of the user in response to the plurality of pressures and the plurality of kinematic data associated with the portions of the body of the user.

According to one aspect, a method for injury reduction is described. One method may include capturing, with a motion capture system comprising a plurality of motion sensors and worn by a user, motion data associated with portions of a body of the user in response to the user performing a plurality of physical actions, and capturing, with a smart insole comprising at least one pressure sensor coupled to a shoe worn by the user, a plurality of pressures associated with a foot of the user in response to the user performing the plurality of physical actions. A process may include determining, in a system coupled to the motion capture system and the smart insole, a plurality of kinematic data associated with the portions of the body of the user in response to the motion data associated with portions of the body and a biomechanical model, and determining, in the system, a plurality of physical loadings associated with joints of the body of the user in response to the plurality of pressures and the plurality of kinematic data associated with the portions of the body of the user.

### BRIEF DESCRIPTION OF THE DRAWINGS

In order to more fully understand the present invention, reference is made to the accompanying drawings. Understanding that these drawings are not to be considered limitations in the scope of the invention, the presently described embodiments and the presently understood best mode of the invention are described with additional detail through use of the accompanying drawings in which:

FIG. 1 illustrates an example configuration according to some embodiments;

FIG. 2 illustrates a process diagram according to various embodiments of the present invention;

FIGS. 3A-C illustrate a process diagram according to additional embodiments of the present invention; and

FIG. 4 illustrates an block diagram according to various embodiments.

### DETAILED DESCRIPTION

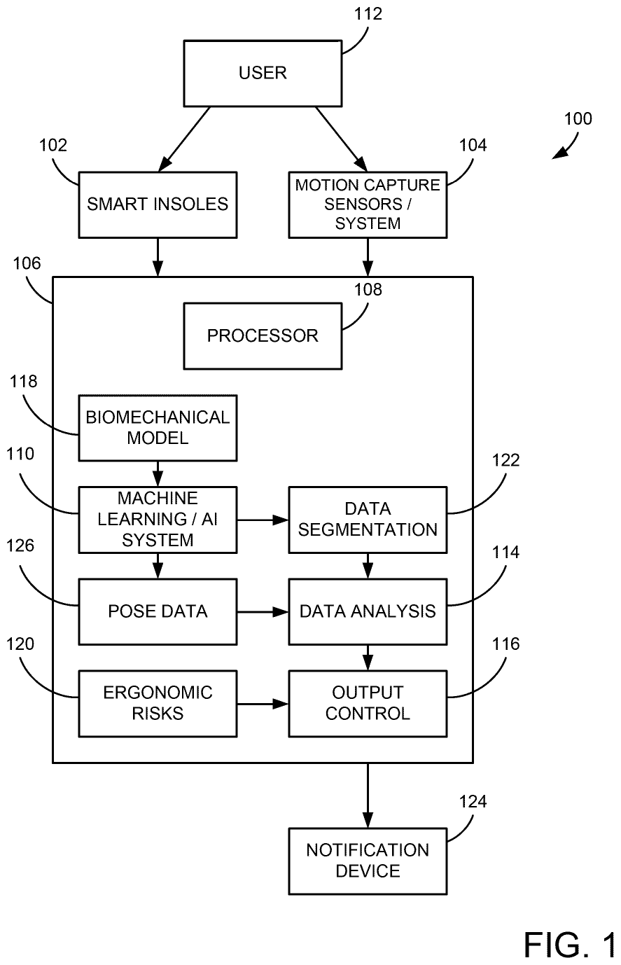

FIG. 1 illustrates a block diagram for some embodiments. More specifically, a system 100 is illustrated including smart insoles 102, motion capture sensors/system 104, and a processing system 106. Processing system 106 may include a memory that stores data and software modules that execute upon a processor 108. Software may include an implementation of a machine learning/AI module 110 to help perform a data segmentation functionality 122 and a pose determination functionality 126. Software may also include functionality that uses pose data 126 of a biomechanical model 118 and captured motion data to perform a data analysis 114 of the user. Still further, software may be included that compares ergonomic risk factors 120 to data analysis 114 and provides feedback or other output 116. A user 112 is shown interacting system 100, e.g. providing input data to, and a notification device 124 is shown receiving output data from system 100.

In some embodiments, smart insoles 102 may include one or more pressure sensors that may be placed within one or more shoes of user 112. In some examples, the pressure sensor may be placed inside the shoe, under the foot of the user, the pressure sensor may be placed on the sole of the shoe, and the like. In other examples, one pressure sensor may be used per shoe, one pressure sensor may be used under the ball of the foot and one pressure sensor may be used under the heel of the foot, and the like. In various embodiments, smart insoles 102 may include a processor for collecting pressure data and a memory for storing the pressure data. Additionally, smart insoles 102 may include a direct (optical or wires) or wireless (e.g. Bluetooth, ultrawide band (UWB)) interface for communicating the pressure data to system 100. In other embodiments, smart gloves or pressure-sensitive gloves may be used alone, or in combination with smart insoles 102

In some embodiments, motion capture sensors/system 104 may include accelerometers, gyroscopes, magnetic sensors, or the like, that may be placed upon different portions of user 112. In some examples, the sensors may be attached to the wrists, spine, head, waist, thighs, knees, ankles, shoulders, pelvis or the like. These sensors may be coupled via directly (optical or wires) or wirelessly (e.g. Bluetooth, UWB, etc.) to a data capture unit which may itself include a processor and memory for storing the captured motion data. In some embodiments, the data capture unit may also process raw data from motion capture sensors 104 to determine kinematic data (e.g. positions, joint angles, and orientation data), or the like of the portions of the user. The data capture unit may in turn provide the motion data or the position and orientation data to processing system 106, via a direct (optical or wires) or wireless (Bluetooth, UWB, etc.) interface. In some embodiments, motion capture sensors 104 may be reflective dots, and a separate motion capture system including multiple cameras and infrared light sources may be used.

One or more processors 108 are illustrated within processing system 106. In some embodiments, processor 108 may receive input data from smart insoles 102 and motion capture sensors 104.

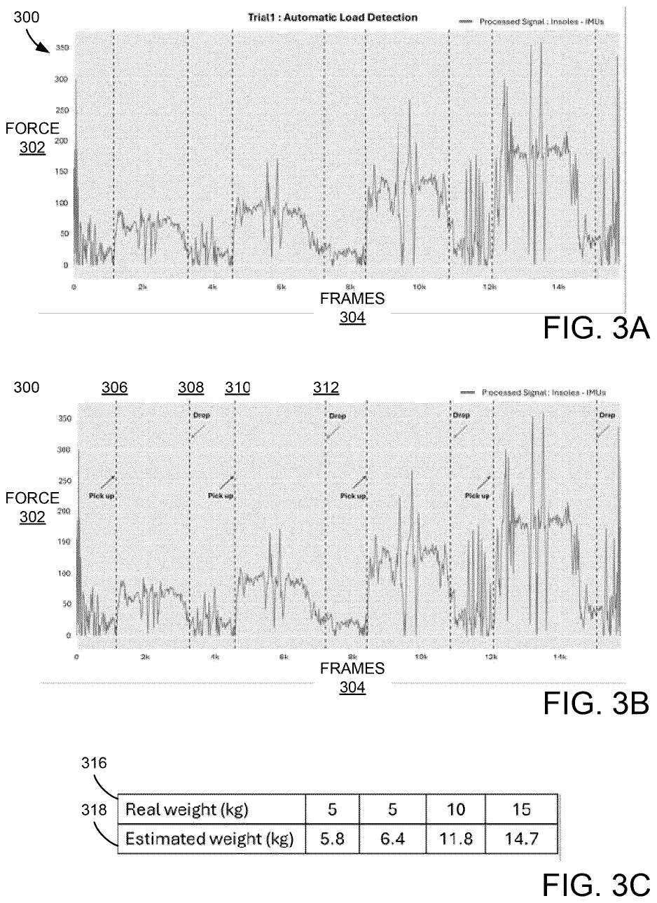

In the example in FIG. 1, software operations may be executed in processor 108 either in series or in parallel threads. In some embodiments, processing system 106 may implement machine learning/AI system 110 to automatically determine data segments 122 and pose data 126. More specifically, AI system 110 may be used to receive the input data and to automatically identify or pick out input data associated with specific movements of the user of interest. It is contemplated that during the period of time user 112 wears insoles 102 and system 104, user 112 may perform multiple repetitions of the same movement, e.g. bending down to lift a box and placing the box on a shelf, or different movements of interest, e.g. bending down to lift the box from a bottom shelf to a higher shelf, picking up an object from a first station and twisting their torso to put the object on a second station, and the like. In either of these cases, the stream of data received and processed by system 100 may be voluminous. As discussed above, a problem is that someone has to monitor the user and indicate what input data is associated with which motion or series of motions. Performing this manually in real-time or even afterwords is very time consuming and imprecise due to some of the factors discussed above. Examples of this are illustrated in FIGS. 3A-B.

In some embodiments, the posing and analysis of biomechanical model 118 based upon input data may also be performed leveraging functionality in AI system 110 based upon the segmented data. In other words, particular sets of motions of user 112 may be determined and separated from other sets of motions.

In this example, a memory of processing system 106 may include a biomechanical model 118 which typically is a software model that simulates the skeleton, movement and constraints corresponding to a typical human body. In various embodiments, the input data (for example, from insoles 102 and system 104) may be mapped to biomechanical model 118 by AI system 110. In other words, biomechanical model 118 may be used to estimate or simulate the physical pose 126 of user 112. Further, based upon poses 126, computations (analysis) may be performed that estimate the loads, forces, and the like on portions of biomechanical model 118. As an example, during a specific action of user 112, if the pressure determined from insoles 102 increases by fifty pounds, and the arms of user 112 are moved downwards (e.g. user 112 has picked up a fifty pound package), biomechanical model 118 may be used to reflect such stresses. Then, in such an example, if the knees of user 112 were bent, biomechanical model 118 may compute or estimate low stresses on the spine. In contrast, if the knees of user 112 were not bent when picking up the load, biomechanical model 118 may compute or estimate much higher stresses on the spine. Accordingly, in some embodiments, data analysis 114 performed upon biomechanical model 118, and the input data such as pose data 126 and segmented data 122 by AI system 110 are used to estimate positions and forces upon other portions of biomechanical model 118

In other embodiments, user 112 and the corresponding biomechanical model 118 may be virtually any physical object (e.g. a robotic arm, a bridge), a vehicle or airplane or portions thereof, an animal, or the like.

In some embodiments, the memory may also include biomechanical risk factors, e.g. ergonomic risks factors 120. Some specific examples relate to ergonomic assessment work-sheets in the form of a series of lifting equations that consider frequency of movement, distance of movement, weight of physical loads, and the like related to worker safety, or the like.

In various embodiments, additional processes running upon processor 108 may be used to compare the motions and forces of biomechanical model 118 to a series of ergonomic risk factors 120. As examples, ergonomic risks may indicate a desired maximum of forces for different portions of a user at one time, a desirable maximum of forces for portions of a user for repetitive motions, a desirable safe range of shoulder to body angles, a limit on the number of repetitions, duration or any other measure quantifying the repetitive motions, and the like. In light of the present disclosure, one of ordinary skill in the art will readily contemplate other such ergonomic risks.

In some embodiments, if particular estimated motions and forces corresponding to biomechanical model 118 exceed thresholds, processing system 106 may send notice to notification device 124. In some embodiments, notification device may be another computer, a smart phone, tablet, smart watch, wearable device, or the like. In some cases, in response to such notices, a user, e.g. a researcher, may manually indicate that changes to the user's actions may be warranted. For example, the user may develop a worker training program on how to work with reduced injury, the user may re-engineer a work environment to reduce ergonomic risks, the user may specify time limits on certain activities or specify mandatory break times, or the like. In other cases, a user, e.g. the worker being monitored, may be advised how to move to avoid the ergonomic risks. For example, the user may be warned not to lift such heavy weights, the user may be advised to take a break, the user may be advised how to move with reduced injury risk, or the like.

In additional embodiments, pressure sensors in the form of smart gloves may also be used. These smart gloves may be used to sense the amount of load placed on the user's hands as they lift an object, carry and object, throw an object, and the like. In such embodiments, this pressure data may also be input into biomechanical model 118 and be used to estimate the stresses on different portions of the user and/or biomechanical model 118.

In light of the above, many other types of data capture, processing architectures, and feed-back mechanisms are contemplated.

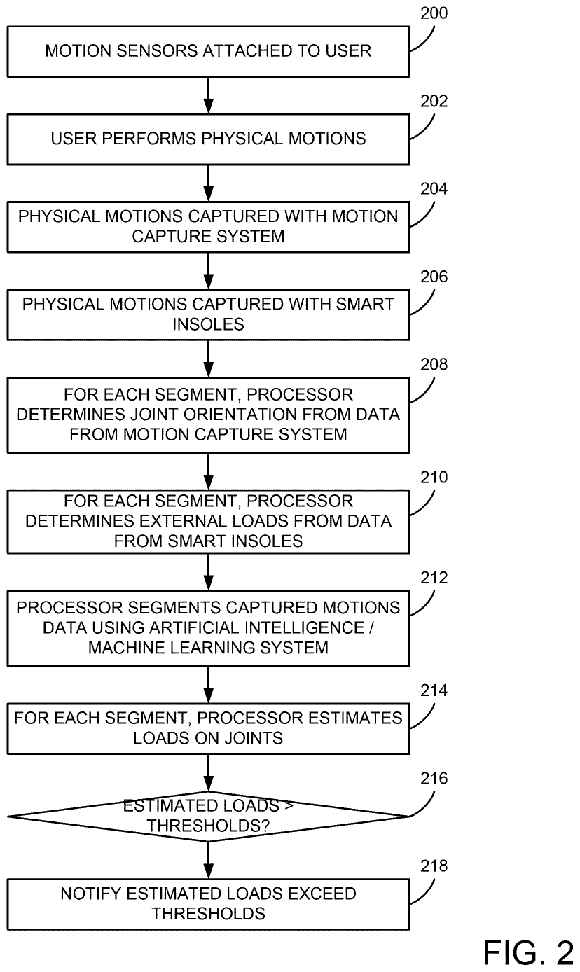

FIG. 2 illustrates a flow diagram according to various embodiments. More specifically, FIG. 2 illustrates a process for capturing, processing and monitoring user movement data. For sake of convenience, reference to elements in FIG. 1 are provided. It should be understood that many other types of data acquisition and processing architectures may be used in other embodiments.

Initially, user 112 is equipped with motion capture hardware, step 200. In some examples, a user may strap on multiple motion capture units to specific portions of their body, or put on clothing having such motion capture units already attached thereto. In some embodiments, sensors including accelerometers, gyroscopes, magnetometers, and the like from Xsens and Movella, the assignee of the present invention, may be utilized. In other embodiments, MEMS sensors from other vendors may also be used. In still other embodiments, optical motion tracking systems may be used, wherein the user wears clothing having reflective dots, or the like.

In various embodiments, in step 200, user 112 may also wear pressure sensors on their shoes. In some embodiments, the sensors may be on the soles and/or the heels of the shoes, and in other embodiments, the pressure sensors may be in the form of insoles that are placed with the user's shoes, or the like. In some embodiments, a single pressure sensor is placed in each shoe, and in other embodiments, multiple pressure sensors are used, for example, one for the heel and one for the ball of the foot.

Next, in FIG. 2, user 112 may perform their duties, including performing specific motions or sets of motions, step 202. In some embodiments, the user may be at their work station or a model work station and may be asked to perform specific motions, e.g. lifting a box from a conveyer belt, placing it upon a scale, and placing it upon a specific shelf based upon its weight. In a model work station, often the user may be asked to perform such actions a number of times, then the user may be asked to perform another action a number of times, etc. In other embodiments, in this step, the user may be at their work station and may be asked to perform their normal duties. In still other embodiments a simulated work station may include use of a virtual reality (VR) system, augmented reality (AR) system, or the like.

In various embodiments, while the user performs their actions, motion capture system 104, captures the user's physical motions, step 204. As mentioned above, the motion capture system 104 may include accelerometers, gyroscopes, magnetometers, or the like, and each of these sensors capture data as the user moves about. In some embodiments, the raw data may be sent to a centralized data capture unit and then onto processing system 106. In other embodiments, the raw data may be processed by the centralized data capture unit to determine kinematic data (e.g. joint angles, positions and orientations) which is then output to processing system 106. As an example of this, the centralized data capture system may receive the data from the motion capture devices and then process the data to determine the kinematic data associated with the user's forearm with respect to gravity and the rotation angles of the wrist; the system may process the data and determine a twist or a bend of a spine; and the like.

In various embodiments, while the user performs their actions, smart insoles 102 (and/or smart gloves), also captures pressures applied by the feet of the user, step 206. In various embodiments, smart insoles 102 may provide raw pressure data to processing system 106; smart insoles 102 may process the raw pressure data and provide weight data to processing system 106; or the like. In some examples, as the user stands still, the pressures sensed by smart insoles 102 are typically symmetric; as the user walks unladen, the pressures sensed will shift over time; as the user carries a load, the amount of pressure sensed will typically increase by the weight of the load, and the like. Additionally, in cases where there are multiple pressure sensors in each smart insoles 102, smart insoles 102 may provide data indicating whether the user is leaning forward (e.g. higher pressure on the ball of the foot versus the heel of the foot), whether the user is leaving backward (e.g. lower pressure on the ball of the foot versus the heel of the foot), and the like. In some embodiments, smart insoles 102 may also include accelerometers, gyroscopes, relative positioning sensors (e.g. ultrawide band sensors), and the like for capturing additional movement characteristics of the foot of the user.

Next, processing system 106 may determine kinematic data (e.g. joint angles, orientations, and positions) for a biomechanical model of the user, based upon the motion data, step 208. As discussed above, embodiments include biomechanical model 118 that reflects the same geometric structure as well as movement constraints of user 112. In various embodiments, the movement data captured in step 204, above, may include the orientations of motion capture devices (e.g. including accelerometers and gyroscopes) that provide the position of the device with respect to gravity, movement and joint angles of the device with respect to pivot locations, include kinematic data of motion capture devices, positions of the devices and their orientation with respect to gravity, and the like. In some example, the data is mapped to a biomechanical model of the user, and then the processing system solves for pose (segment orientation, position) of the user. In some embodiments, as a result of this step, for a specific segment of time, biomechanical model 118 may be posed and moved.

In various embodiments, processing system 106 may then determine what external loads user 112 is being subject to, step 210. As discussed above, during a training phase, system 110 may receive pressures from smart insoles 102 while user 112 walks or stands unladen. In this step, such data may be compared to the pressures of smart insoles 102 while performing a (segmented) action. The comparisons may thus indicate how much of an external load the user is being subjected to while performing this action. For example, a determination may be made that the user is subject to an extra 50 pound load (e.g. the user is carrying a 50 pound box).

In various embodiments, processing system 106 may automatically segment the motion data from motion capture system 104 and pressure data from smart insoles 102 (and/or smart gloves or pressure-sensitive gloves), step 212. More specifically, processing system 106 may include an instance of a machine learning/AI system 110 that will automatically determine time periods from the incoming data which are associated with specific user actions. It is contemplated that system 110 may be trained by providing it with multiple instances of motion and pressure data of a worker performing multiple instances of a first specific action, then providing it with multiple instances of motion and pressure data of the worker performing multiple instances of a second specific action, and the like. By doing so, system 110 may then be trained to determine the specific action being performed by the worker, based upon the motion and pressure data. As described above, some embodiments may be used to capture data from a worker at their normal work station throughout the day or for an extended period of time. Accordingly, in this step, system 110 may automatically segment the stream of motion and pressure data for analysis, based upon the actions that are recognized. For example, system 110 may sense a worker performs in time: a dead lifting action, a walking with a load action, a lifting a load to an over-head location, a walking action, a squatting pull action, and the like, based upon the motion and pressure data, or the like. In other embodiments, segmentation of the motion data and the pressure data may occur at other times within FIG. 2.

Subsequently, in various embodiments, processing system 106 may determine inferred loads being placed upon various portions of biomechanical model 118, based upon the external load determined in step 210, step 214. More specifically, in some embodiments, the external load may be assumed to be placed upon the hands of a user, thus the hands of biomechanical model 118 may be assigned a force downwards associated with the external load. Subsequently, the external load may be distributed throughout biomechanical model 118. For example, the load on the hands are transferred to the shoulders, via the elbow joints, the load on the shoulders are transferred to the hips, via the spine, the weight on the hips are transferred through to the feed, via the knee joints, and the like. In various embodiments, these load transfer computations may be performed for biomechanical model 118 throughout the period of time associated with a segmented motion. In various embodiments, the loads may refer to joint loads of a user, as well as physical loads carried by the user.

In various embodiments, the inferred loads (e.g. the loads, actions, duration, etc.) for different portions of biomechanical model 118 may then be compared to movement/load thresholds, step 216. As discussed above, a number of ergonomic risk factors may be used that indicate risk of injury based upon loads, durations, actions (e.g. twist, lift), and the like. For example, an ergonomic risk factor may indicate that user should not carry a load that exceeds 75 pounds, above waist high for over 2 seconds, another risk factor may indicate that a user should not carry a 50 pound load above their shoulders, another risk factor may indicate a threshold on a number of repetitions for a repetitive action with low or no weight is exceeded, another risk factor may include the user staying in a certain pose over a threshold amount of time, and the like. In some embodiments, multiple risk factors (e.g. limb angle, limb orientation, inferred loads, etc.) from different portions of biomechanical model 118 (e.g. neck, shoulder, knee, torso, elbow, wrist, etc.) may be evaluated together to determine a risk factor for the worker actions.

In some embodiments, if risk factors are exceeded for the particular action, processing system 106 may output an exception to notification device 124, step 218. The notice may be a message to a computer, smart phone, smart watch, tablet, or the like. In some embodiments, the notice may go to a motion planner, or the like. That planner may then suggest a revised series of motions for the user to perform to reduce the risks. In other embodiments, the notice may be provided in near-real time to the worker. For example, a notice may go to a smart watch of the worker, and indicate which of their motions is ergonomically risky and what they can do to reduce their injury risk, and the like. Accordingly, this step may provide feedback that reduces injury risks of the user/worker.

FIGS. 3A-C illustrate experimental results of an embodiment. More specifically, the chart 300 in FIG. 3A represents force or pressure 302 as a function of time, e.g. frames 304. In this experiment, the force is derived from smart insoles disposed within a user's shoe, while the user performed a number of different tasks. Specifically, the user picked-up weights with different weights.

In some embodiments, the amount of force may be processed or filtered to reduce the amount of short-term or transient forces. Then, as illustrated in FIG. 3B, based upon the processed data, events, e.g. 306, 308, 310, 312, etc. may be determined. These events may indicate a time when a user motion begins, when the user motion ends, when the next user motion begins, and the like. As annotated in FIG. 3B, time 306 is when a user begins to pick up a weight, and that lasts until time period 308; time 310 is when the user begins to pick up another weight, and that lasts until time period 312, and the like. In other words between time period 306-308, the user carries a first weight; between time period 310-312, the user carries a second weight; and the like

In various embodiments, FIG. 3C illustrates initial experimental results of the actual weight 316 carried by the user compared to a computed load 318. According to the events determined in FIG. 3B, the data illustrated in FIG. 3A may be segmented, and the segmented data may be used for various embodiments, described above, to compute loads placed upon different portions of the user's body. In this example, the segmented data between time periods 306-308 is processed to estimate that the user picks up a 5.8 kilogram weight; and the segmented data between time periods 310-312 is processed to estimate that user picks up a 6.4 kilogram weight. As can be seen in FIG. 3C, the computed loads in 318 approximately correlate with the actual weights 316. With further experimentation, the accuracy in computed loads 318 compared to actual loads 316 are expected to increase.

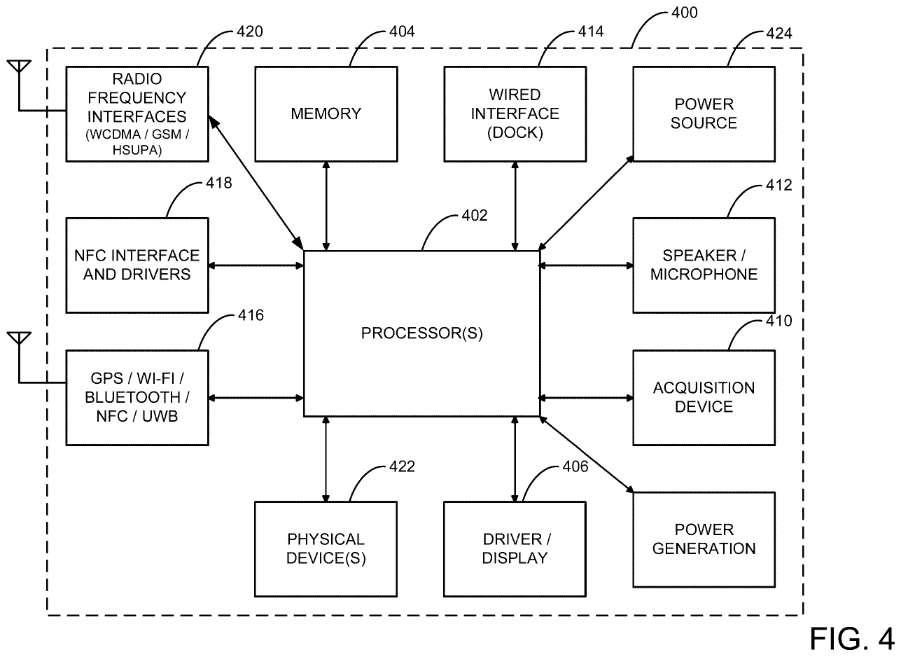

FIG. 4 illustrates a functional block diagram of various embodiments of the present invention. More specifically, it is contemplated that user smart devices (e.g. smart devices, smart phones, tablets), computers (e.g. servers, laptops, streaming servers, viewer systems, provider servers, advertisement servers, streaming client system, etc.), processing units, etc. may be implemented with a subset or superset of the below illustrated components.

In FIG. 4, a computing device 400 may include some, but not necessarily all of the following components: an applications processor/microprocessor 402, memory 404, a display 406, an image acquisition device 410, audio input/output devices 412, and the like. Data and communications from and to computing device 400 can be provided by via a wired interface 414 (e.g. Ethernet, dock, plug, controller interface to peripheral devices); miscellaneous rf receivers, e.g. a GPS/Wi-Fi/Bluetooth interface/UWB 416; an NFC interface (e.g. antenna or coil) and driver 418; RF interfaces and drivers 420, and the like. Also included in some embodiments are physical sensors 422 (e.g. (MEMS-based) accelerometers, gyros, magnetometers, pressure sensors, temperature sensors, bioimaging sensors etc.).

In various embodiments, computing device 400 may be a computing device (e.g. Apple iPad, Microsoft Surface, Samsung Galaxy Note, an Android Tablet); a smart phone (e.g. Apple iPhone, Google Pixel, Samsung Galaxy S); a portable computer (e.g. netbook, laptop, convertible), a media player (e.g. Apple iPod); a reading device (e.g. Amazon Kindle); a fitness tracker (e.g. Fitbit, Apple Watch, Garmin or the like); a headset or glasses (e.g. Apple Vision Pro, Meta Quest, HTC Vive, Sony PlaystationVR); a wearable device (e.g. Motiv smart ring, smart headphones); an implanted device (e.g. smart medical device), a point of service (POS) device, a server, or the like. Typically, computing device 400 may include one or more processors 402. Such processors 402 may also be termed application processors, and may include a processor core, a video/graphics core, and other cores. Processors 402 may include processor from Apple (A14 Bionic, A15 Bionic), NVidia (Tegra), Intel (Core), Qualcomm (Snapdragon), Samsung (Exynos), ARM (Cortex), MIPS technology, a microcontroller, and the like. In some embodiments, processing accelerators may also be included, e.g. an AI accelerator, Google (Tensor processing unit), a GPU, or the like. It is contemplated that other existing and/or later-developed processors/microcontrollers may be used in various embodiments of the present invention.

In various embodiments, memory 404 may include different types of memory (including memory controllers), such as flash memory (e.g. NOR, NAND), SRAM, DDR SDRAM, or the like. Memory 404 may be fixed within computing device 400 and may also include re-movable memory (e.g. SD, SDHC, MMC, MINI SD, MICRO SD, SIM). The above are examples of computer readable tangible media that may be used to store embodiments of the present invention, such as computer-executable software code (e.g. firmware, application programs), security applications, application data, operating system data, databases or the like. Additionally, in some embodiments, a secure device including secure memory and/or a secure processor are provided. It is contemplated that other existing and/or later-developed memory and memory technology may be used in various embodiments of the present invention.

In various embodiments, display 406 may be based upon a variety of later-developed or current display technology, including LED or OLED displays and/or status lights; touch screen technology (e.g. resistive displays, capacitive displays, optical sensor displays, electro-magnetic resonance, or the like); and the like. Additionally, display 406 may include single touch or multiple-touch sensing capability. Any later-developed or conventional output display technology may be used for embodiments of the output display, such as LED IPS, OLED, Plasma, electronic ink (e.g. electrophoretic, electrowetting, interferometric modulating), or the like. In various embodiments, the resolution of such displays and the resolution of such touch sensors may be set based upon engineering or non-engineering factors (e.g. sales, marketing). In some embodiments, display 406 may integrated into computing device 400 or may be separate. In some embodiments, display 406 may be in virtually any size or resolution, such as a 4K resolution display, a microdisplay, one or more individual status or communication lights, e.g. LEDs, or the like.

In some embodiments of the present invention, acquisition device 410 may include one or more sensors, drivers, lenses and the like. The sensors may be visible light, infrared, and / or UV sensitive sensors, ultrasonic sensors, or the like, that are based upon any later-developed or convention sensor technology, such as CMOS, CCD, or the like. In some embodiments of the present invention, image recognition algorithms, image processing algorithms or other software programs for operation upon processor 402, to process the acquired data. For example, such software may pair with enabled hardware to provide functionality such as: facial recognition (e.g. Face ID, head tracking, camera parameter control, or the like); fingerprint capture/analysis; blood vessel capture/analysis; iris scanning capture/analysis; otoacoustic emission (OAE) profiling and matching; and the like. In additional embodiments of the present invention, acquisition device 410 may provide user input data in the form of a selfie, biomechanical data, or the like.

In various embodiments, audio input/output 412 may include a microphone(s)/speakers. In various embodiments, voice processing and/or recognition software may be provided to applications processor 402 to enable the user to operate computing device 400 by stating voice commands. In various embodiments of the present invention, audio input 412 may provide user input data in the form of a spoken word or phrase, or the like, as described above. In some embodiments, audio input/output 412 may be integrated into computing device 400 or may be separate.

In various embodiments, wired interface 414 may be used to provide data or instruction transfers between computing device 400 and an external source, such as a computer, a remote server, a POS server, a local security server, a storage network, another computing device 400, an IMU, video camera, a motion capture system, or the like. Embodiments may include any later-developed or conventional physical interface/protocol, such as: USB, micro USB, mini USB, USB-C, Firewire, Apple Lightning connector, Ethernet, POTS, custom interface or dock, or the like. In some embodiments, wired interface 414 may also provide electrical power, or the like to power source, or the like. In other embodiments interface 414 may utilize close physical contact of device 400 to a dock for transfer of data, magnetic power, heat energy, light energy, laser energy or the like. Additionally, software that enables communications over such networks is typically provided.

In various embodiments, a wireless interface 416 may also be provided to provide wireless data transfers between computing device 400 and external sources, such as computers, storage networks, headphones, microphones, cameras, IMUs or the like. As illustrated in FIG. 4, wireless protocols may include Wi-Fi (e.g. IEEE 802.11 a/b/g/n, WiMAX), Bluetooth, Bluetooth Low Energy (BLE) IR, near field communication (NFC), ZigBee, Ultra-Wide Band (UWB), Wi-Fi, mesh communications, and the like.

GNSS (e.g. GPS) receiving capability may also be included in various embodiments of the present invention. As illustrated in FIG. 4, GPS functionality is included as part of wireless interface 416 merely for sake of convenience, although in implementation, such functionality may be performed by circuitry that is distinct from the Wi-Fi circuitry, the Bluetooth circuitry, and the like. In various embodiments of the present invention, GPS receiving hardware may provide user input data in the form of current GPS coordinates, or the like, as described above.

Additional wireless communications may be provided via RF interfaces in various embodiments. In various embodiments, RF interfaces 420 may support any future-developed or conventional radio frequency communications protocol, such as CDMA-based protocols (e.g. WCDMA), GSM-based protocols, HSUPA-based protocols, G4, G5, or the like. In some embodiments, various functionality is provided upon a single IC package, for example the Marvel PXA330 processor, and the like. As described above, data transmissions between a smart device and the services may occur via Wi-Fi, a mesh network, 4G, 5G, or the like.

Although the functional blocks in FIG. 4 are shown as being separate, it should be understood that the various functionality may be regrouped into different physical devices. For example, some processors 402 may include Bluetooth functionality. Additionally, some functionality need not be included in some blocks, for example, GPS functionality need not be provided in a provider server.

In various embodiments, any number of future developed, current operating systems, or custom operating systems may be supported, such as iPhone OS (e.g. iOS), Google Android, Linux, Windows, MacOS, or the like. In various embodiments of the present invention, the operating system may be a multi-threaded multi-tasking operating system. Accordingly, inputs and/or outputs from and to display 406 and inputs/or outputs to physical sensors 422 may be processed in parallel processing threads. In other embodiments, such events or outputs may be processed serially, or the like. Inputs and outputs from other functional blocks may also be processed in parallel or serially, in other embodiments of the present invention, such as acquisition device 410 and physical sensors 422.

In some embodiments of the present invention, physical sensors 422 (e.g. MEMS-based) may include accelerometers, gyros, magnetometers, pressure sensors, temperature sensors, ECG sensors, imaging sensors (e.g. blood oxygen, heartbeat, blood vessel, iris data, etc.), thermometer, otoacoustic emission (OAE) testing hardware, and the like. The data from such sensors may be used to capture data associated with device 400, and a user of device 400. Such data may include physical motion data, pressure data, orientation data, or the like. Data captured by sensors 422 may be processed by software running upon processor 402 to determine characteristics of the user, e.g. gait, gesture performance data, or the like and used for user authentication purposes. In some embodiments, sensors 422 may also include physical output data, e.g. vibrations, pressures, and the like.

In some embodiments, a power supply 424 may be implemented with a battery (e.g. LiPo), ultracapacitor, or the like, that provides operating electrical power to device 400. In various embodiments, any number of power generation techniques may be utilized to supplement or even replace power supply 424, such as solar power, liquid metal power generation, thermoelectric engines, rf harvesting (e.g. NFC) or the like.

FIG. 4 is representative of components possible for a processing device. It will be readily apparent to one of ordinary skill in the art that many other hardware and software configurations are suitable for use with the present invention. Embodiments of the present invention may include at least some but need not include all of the functional blocks illustrated in FIG. 4. For example, a smart phone (e.g. processing unit/video camera) may include some, but not all of the illustrated functionality. As additional examples, a motion capture system, may include some of the functional blocks in FIG. 4, but it need not include a speaker or microphone; a processing server need not include global positioning capability; a smart insole need not include a display; or the like.

Further embodiments can be envisioned to one of ordinary skill in the art after reading this disclosure. For example, different types of motion capture systems may be used, including optical motion capture and reflectors, video motion capture, and the like. In still other embodiments, motion capture systems may include ultrasonic sensors, ultrawide-band technologies, and the like. Additionally, many different types of pressure sensors may be used in various embodiments for smart insoles or smart gloves, such as piezo-electric sensors, strain gauges, MEMS sensors, and the like.

Other embodiments may also be applied to applications other than user motion analysis and assessment. For example, embodiments may include monitoring motion of machines, robotic arms, vehicle components, and the like. Similar to the above, motion data, pressure data, magnetic data, and the like measured on an object may be input into a system which also stores a physical model of the object. Then, based upon these data, computed forces, pressures, strains, and the like may be computed for virtually all other portions of the object model. Next, the computed forces may be compared to thresholds (e.g. set forces, number or repetitions, duration, etc.) to determine possible failures of portions of the object, and possible ways to mitigate or reduce these failures by changing the movement and loads placed upon the object.

The block diagrams of the architecture and flow charts are grouped for ease of understanding. However, it should be understood that combinations of blocks, additions of new blocks, re-arrangement of blocks, and the like are contemplated in alternative embodiments of the present invention. The specification and drawings are, accordingly, to be regarded in an illustrative rather than a restrictive sense. It will, however, be evident that various modifications and changes may be made thereunto without departing from the broader spirit and scope of the invention as set forth in the claims.

We claim:

1. A system for injury reduction comprises:
a motion capture system worn by a user comprising a plurality of motion sensors, wherein the motion capture system is configured to capture motion data associated with portions of a body of the user in response to the user performing a plurality of physical actions;
a smart insole coupled to a shoe worn by the user, comprising at least one pressure sensor, wherein the one pressure sensor is configured to measure a plurality of pressures associated with a foot of the user in response to the user performing the plurality of physical actions; and
a system coupled to the motion capture system and the smart insole, wherein the system is configured to store a biomechanical model of the user, wherein the system is configured to determine a plurality of kinematic data associated with the portions of the body of the user in response to the motion data associated with portions of the body and the biomechanical model, wherein the system is configured to determine a plurality of physical loadings associated with joints of the body of the user in response to the plurality of pressures and the plurality of kinematic data associated with the portions of the body of the user.

2. The system of claim 1
wherein plurality of physical actions comprises a first physical action followed by a second physical action;
wherein the system is configured to determine a first subset of kinematic data from the plurality of kinematic data and a second subset of kinematic data from the plurality of kinematic data in response to the motion data;
wherein the first subset of kinematic data is associated with the first physical action; and
wherein the second subset of kinematic data is associated with the second physical action.

3. The system of claim 2 wherein the first physical action is associated with an action selected from a group consisting: lifting an object, twisting a torso of the user, walking, squatting, the user standing on tippy toes and standing.

4. The system of claim 2 wherein the system is configured to determine a first subset of physical loadings from the plurality of physical loadings in response to the plurality of pressures and the first subset of kinematic data.

5. The system of claim 1
wherein the smart insole comprises a first pressure sensor and second pressure sensor;
wherein the first pressure sensor is associated with a forward portion of the foot of the user; and
wherein the second pressure sensor is associated with a rearward portion of the foot of the user.

6. The system of claim 1 further comprising:
a smart glove coupled to a hand of the user, comprising at least one pressure sensor, wherein the one pressure sensor is configured to measure a plurality of pressures associated with the hand of the user in response to the user performing the plurality of physical actions; and
wherein the system is configured to determine the plurality of physical loadings associated with joints of the body of the user in response to the plurality of pressures, the plurality of pressures and the plurality of kinematic data associated with the portions of the body of the user.

7. The system of claim 1
wherein the system is configured to determine whether the plurality of physical loadings exceeds a threshold of physical loadings; and
wherein the system is configured to direct output of corrective physical actions in response to determining that the plurality of physical loadings exceeds the threshold of physical loadings.

8. The system of claim 7
wherein the plurality of physical actions is associated with a time duration;
wherein the system is configured to determine whether the time duration exceeds a duration threshold; and
wherein the system is configured to direct output of corrective physical actions in response to determining that the plurality of physical loadings exceeds the threshold of physical loadings and in response to determining that the time duration exceeds the duration threshold.

9. The system of claim 8
wherein the corrective physical actions are selected from a group consisting of:
reducing an amount of weight carried by the user, reducing an amount of twisting of the user, reducing the time duration by the user, changing a posture of the user.

10. The system of claim 1
wherein the plurality of motion sensors are selected from a group consisting of: a three-dimensional sensor, an accelerometer, a gyroscope and a magnetometer.

11. A method for injury reduction comprises:
capturing, with a motion capture system comprising a plurality of motion sensors and worn by a user, motion data associated with portions of a body of the user in response to the user performing a plurality of physical actions;
capturing, with a smart insole comprising at least one pressure sensor coupled to a shoe worn by the user, a plurality of pressures associated with a foot of the user in response to the user performing the plurality of physical actions;
determining, in a system coupled to the motion capture system and the smart insole, a plurality of kinematic data associated with the portions of the body of the user in response to the motion data associated with portions of the body and a biomechanical model; and
determining, in the system, a plurality of physical loadings associated with joints of the body of the user in response to the plurality of pressures and the plurality of kinematic data associated with the portions of the body of the user.

12. The method of claim 11
wherein plurality of physical actions comprises a first physical action followed by a second physical action; and
wherein the method further comprises determining, in the system, a first subset of kinematic data from the plurality of kinematic data and a second subset of kinematic data from the plurality of kinematic data in response to the motion data, wherein the first subset of kinematic data is associated with the first physical action, and wherein the second subset of kinematic data is associated with the second physical action.

13. The method of claim 12 wherein the first physical action is associated with an action selected from a group consisting: lifting an object, twisting a torso of the user, walking, squatting, the user standing on tippy toes and standing.

14. The method of claim 12 further comprising: determining, in the system, a first subset of physical loadings from the plurality of physical loadings in response to the plurality of pressures and the first subset of kinematic data.

15. The method of claim 11
wherein the smart insole comprises a first pressure sensor associated with a forward portion of the foot of the user and second pressure sensor associated with a rearward portion of the foot of the user;
wherein the method further comprises:
capturing, with the first pressure sensor, a first set of pressures of the foot of the user performing the plurality of physical actions; and
capturing, with the second pressure sensor, a second set of pressures of the foot of the user performing the plurality of physical actions; and
wherein the plurality of pressures comprise the first set of pressures and the second set of pressures.

16. The method of claim 11 further comprising:
capturing, with a smart glove comprising at least one load sensor coupled to a hand of the user, a plurality of loads associated with the hand of the user in response to the user performing the plurality of physical actions; and
wherein determining, in the system, the plurality of physical loadings associated with joints of the body of the user is in response to the plurality of pressures, the plurality of loads, and the plurality of kinematic data associated with the portions of the body of the user.

17. The method of claim 11 further comprising:
determining, with the system, whether the plurality of physical loadings exceeds a threshold of physical loadings; and
directing, with the system, output of corrective physical actions in response to determining that the plurality of physical loadings exceeds the threshold of physical loadings.

18. The method of claim 17
wherein the plurality of physical actions is associated with a time duration;
wherein the method further comprises determining, with the system, whether the time duration exceeds a duration threshold; and
wherein the directing, with the system, output of corrective physical actions is in response to determining that the plurality of physical loadings exceeds the threshold of physical loadings, and in response to determining that the time duration exceeds the duration threshold.

19. The method of claim 18
wherein the corrective physical actions are selected from a group consisting of: reducing an amount of weight carried by the user, reducing an amount of twisting of the user, reducing the time duration by the user, changing a posture of the user.

20. The method of claim 11
wherein the plurality of motion sensors are selected from a group consisting of: a three-dimensional sensor, an accelerometer, a gyroscope, and a magnetometer.
